(19) United States
(12) Patent Application Publication
(10) Pub. No.: US 2008/0206172 A1
(43) Pub. Date: Aug. 28, 2008

(54) CLEAR SUNSCREEN GELS AND METHODS OF USE THEREOF

(76) Inventors: Fatemeh Mohammadi, Hauppauge, NY (US); Anna Czarnota, Huntington, NY (US); Bassi-Sarna Manju, Farmingdale, NY (US)

Correspondence Address: THE ESTEE LAUDER COS, INC, 155 PINELAWN ROAD, STE 345 S, MELVILLE, NY, 11747, US

(21) Appl. No.: 11/679,269

(22) Filed: Feb. 27, 2007

(51) Int. Cl. A61Q 17/04; A61K 8/72

(52) U.S. Cl. 424/60; 424/59

(57) ABSTRACT

The present invention relates to an alcohol-free transparent sunscreen composition containing at least one chemical ultra-violet (UV) light absorber, at least one solvent and at least one diphenyl silicone elastomer gellant. Preferably, the solvent is selected from the group consisting of dimethicone, dicaprylyl carbonate, lauryl lactate, neopentyl glycol diheptanoate, glycerin, and combinations thereof, and the diphenyl silicone elastomer gellant contains diphenylsiloxy phenyl trimethicone. Such a transparent sunscreen composition can be used to form a stable and aesthetically appeal clear gel for protecting skin against harmful effects of UV radiation.

### FIELD OF THE INVENTION

The present invention relates to skin care cosmetic compositions and methods of using the same. In particular, the present invention relates to novel clear, alcohol-free sunscreen gels and methods of use thereof.

### BACKGROUND OF THE INVENTION

The negative effects of exposure to sunlight are well known. For example, prolonged exposure to sunlight can cause short term damage such as sunburn and premature aging of the skin when skin is exposed to UV radiation having a wavelength of from about 290 nm to abut 400 nm. Moreover, long term damage can lead to serious conditions such as skin cancer. As a result of such health hazards caused by excessive sun exposure, sunscreen products continue to be in demand. Therefore, the market provides a variety of sunscreen products, including daily wear products which contain sunscreens. With the increased use of such sunscreen products, consumer demands require the development of sunscreen product that are not only effective shields against the sun's rays, but also are aesthetically pleasing upon application to the skin.

Clear cosmetic compositions are commonly used for antiperspirants and hair gels. Such compositions typically comprise polyols and silicone emulsifiers whose refractive indices are matched to provide transparent emulsions, otherwise known as gels. However, in conventional clear sunscreens, the silicone elastomers normally used do not thicken the sunscreen component sufficiently, so that additional waxes are needed. Unfortunately, the additional waxes destroy the clarity of the conventional clear sunscreen compositions and result in compositions that are often too thick and therefore unappealing to the consumer. Moreover, the conventional sunscreen compositions typically contain alcohols, which may cause irritation and dry the skin upon application of the sunscreen products.

Therefore, there remains a need for improved clear sunscreen gel compositions that have sufficient thickness but at the same time maintain the desired clarity and are aesthetically pleasing to the consumer. Further, there is a need for improved clear sunscreen gel compositions that are essentially free of alcohol and therefore non-irritating to the skin.

### SUMMARY OF THE INVENTION

The present invention at one aspect relates to a cosmetic composition comprising at least one chemical ultra-violet (UV) light absorber, at least one solvent and at least one diphenyl silicone elastomer gellant, wherein the composition is transparent and is essentially free of alcohol.

The term “transparent” as used herein refers to a composition or material having a refractive index (RI) that matches the RI of its surroundings, i.e., such a composition or material is transparent within and relative to its functional environment. Preferably, a transparent composition or material as described by the present invention exhibits a light transmission value of greater than about 70%, more preferably greater than about 75%, and will not exhibit any light scattering property.

The term “essentially free of alcohol” as used herein refers to a composition or material having an ethyl alcohol content of less than about 0.5% by total weight of the composition or material.

At another aspect, the present invention relates to a method of using the above-described transparent cosmetic composition for protecting skin against harmful effects of UV radiation, by applying such a transparent cosmetic composition to the skin.

Other aspects and objectives of the present invention will become more apparent from the ensuring description, examples, and claims.

### DETAILED DESCRIPTION OF THE INVENTION AND PREFERRED EMBODIMENTS THEREOF

Except in operating and comparative examples, or where otherwise explicitly indicated, all numbers in this description indicating amounts or ratios of material or conditions of reaction, physical properties of materials and/or use are to be understood as modified by the word “about.” All amounts are by weight of the final composition, unless otherwise specified.

In the present invention, it has been surprisingly found that by combining a specific diphenyl silicone elastomer gellant and one or more specific non-alcohol solvent(s) with a chemical UV light absorber, a stable, alcohol-free, and transparent cosmetic composition can be formed, which is particularly useful as an aesthetically appealing clear sunscreen gel. The term “stable” as used herein refers to a composition having sufficient physical and chemical stability so that the composition does not separate or precipitate when all of the components are added. The term “aesthetically appealing” as used herein refers to a composition having a powdery, velvety, and silky texture upon application onto the skin.

As a first essential component of the transparent cosmetic composition of the present invention, a chemical UV light absorber is used. The chemical UV light absorber can be any suitable chemical UV light absorbers commonly used in cosmetic compositions. Specific examples of suitable chemical UV light absorbers include, but are not limited to: benzophenone-3, homosalate, ethylhexyl methoxycinnamate, avobenzone, ethylhexyl salicylate and octocrylene and combinations thereof. Preferably, the chemical UV light absorber is present in the transparent cosmetic composition of the present invention at an amount ranging from about 1% to about 20%, more preferably from about 2% to about 15%, by total weight of the composition. Particularly, the transparent cosmetic composition of the present invention does not include any physical UV absorbers, such as titanium oxide and zinc oxide, which are known to destroy the clarity of the composition.

As a second essential component of the transparent cosmetic composition of the present invention, at least one solvent system is used. The solvent system can comprise any suitable solvent or solvents for solubilizing and carrying the chemical UV light absorber. Specific examples of suitable solvents include, but are not limited to: dimethicone (such as DM-fluid A-6CS available from Shin-Etsu Chemical Co., Ltd. at Tokyo, Japan), dicaprylyl carbonate (such as cetiol® CC available from Cognis Corporation at Cincinnati, Ohio.), lauryl lactate (such as ceraphyl® 31 available from International Specialty Products at Wayne, N.J.), neopentyl glycol diheptanoate (such as Lexfeel® 7 available from Inolex Chemical Co. at Philadelphia, Pa.), glycerin, and combinations thereof. Preferably, but not necessarily, the composition of the present invention comprises a silicone-based solvent, such as dimethicone, which is particularly compatible with the chemical UV light absorbers described hereinabove. The solvent(s) may be present in an amount ranging from about 1% to about 30%, preferably, from about 10% to about 29%, and most preferably from about 15% to about 28%, by total weight of the composition.

It is known that chemical UV light absorbers are mostly in powder form with low clarity. High amounts of ethyl alcohol have been used in conventional sunscreen compositions to enhance solubility of such chemical UV light absorbers. However, sunscreen compositions with high ethyl alcohol content usually causes irritation upon application of the compositions to the skin. In the present invention, it has been surprisingly discovered that a third essential ingredient, i.e., a diphenyl silicone elastomer gellant, and more specifically, a diphenyl silicone elastomer gellant comprising diphenylsiloxy phenyl trimethicone, can be used to effectively enhance the solubility of the chemical UV light absorbers, in the absence of ethyl alcohol. While not wishing to be bound by any theories, it is believed that the diphenyl group of the silicone elastomer gellant interacts with the diphenyl group of the UV absorber and thereby enhancing solubility of the UV absorber in the composition. The total amount of diphenylsiloxy phenyl trimethicone in the sunscreen composition of the present invention may range from about 1% to about 50% by total weight of the composition.

In one specific embodiment of the present invention, the diphenyl silicone elastomer gellant comprises a mixture of diphenylsiloxy phenyl trimethicone and dimethicone/phenyl vinyl dimethicone crosspolymer. Preferably, such a mixture comprises from about 80% to about 90% of diphenylsiloxy phenyl trimethicone and from about 10% to about 20% of dimethicone/phenyl vinyl dimethicone crosspolymer by total weight of the mixture. For example, KSG-18A available from Shin-Etsu Chemical Co., Ltd. at Tokyo, Japan, which is a mixture comprising about 85 wt % of diphenylsiloxy phenyl trimethicone and about 15 wt % of dimethicone/phenyl vinyl dimethicone crosspolymer, is used as the diphenyl silicone elastomer gellant in the compositions of the present invention. If KSG-18A is used in the composition of the present invention as the gellant, it may present in an amount ranging from 35% to about 55%, more preferably about 40% to about 50%, and most preferably from about 44% to about 46%, by total weight of each composition.

In another specific embodiment of the present invention, the diphenyl silicone elastomer gellant comprises a mixture of diphenylsiloxy phenyl trimethicone, dimethicone/phenyl vinyl dimethicone crosspolymer, and methyl trimethicone. Preferably, such a mixture comprises from about 10% to about 15% of diphenylsiloxy phenyl trimethicone, from about 25% to about 35% of dimethicone/phenyl vinyl dimethicone crosspolyrner, and from about 55% to about 60% of methyl trimethicone, by total weight of the mixture. For example, X-21-5652D available from Shin-Etsu Chemical Co., Ltd. at Tokyo, Japan, which is a mixture comprising about 13 wt % of diphenylsiloxy phenyl trimethicone, about 30 wt % of dimethicone/phenyl vinyl dimethicone crosspolymer, and about 57 wt % of methyl trimethicone, is used as the diphenyl silicone elastomer gellant in the compositions of the present invention. If X-21-5652D is used in the composition of the present invention as the gellant, it may present in an amount ranging from about 5% to about 25%, more preferably from about 7% to about 20%, and most preferably from about 10% to about 17% by total weight of the composition.

In yet another embodiment of the present invention, the diphenyl silicone elastomer gellant may comprise a first mixture of diphenylsiloxy phenyl trimethicone and dimethicone/phenyl vinyl dimethicone crosspolymer (e.g., KSG-18A), and a second mixture of diphenylsiloxy phenyl trimethicone, dimethicone/phenyl vinyl dimethicone crosspolymer, and methyl trimethicone (e.g., X-21-5652D), as described hereinabove.

In order to further improve the texture of the transparent cosmetic composition of the present invention, a hybrid silicone powder with a silicone rubber core coated with a silicone resin shell can optionally be added to such composition. Specific examples of hybrid silicone powders suitable for use in the present invention include, but are not limited to the KSP-100 powders available from Shin-Etsu Chemical Co., Ltd. at Tokyo, Japan, which comprise a vinyl dimethicone/methicone silsesquioxane crosspolymer. The KSP-100 hybrid silicone powders provide a uniquely soft, silky feel with good dispersibility/spreadability and reduced agglomeration. If KSP-100 is used in the composition of the present invention, it may present in an amount ranging from about 0.5% to about 10%, more preferably from about 1% to about 5%, by total weight of the composition.

Further, the transparent cosmetic composition of the present invention may optionally comprise a thickener for improving the viscosity of the composition without adversely affecting its clarity and aesthetical appeal to customers. Preferably, the thickener comprises a dimethicone/phenyl vinyl dimethicone crosspolymer. More preferably, the thickener comprises a mixture of the dimethicone/phenyl vinyl dimethicone crosspolymer with phenyl trimethicone, such as the KSG-18 silicone paste available from Shin-Etsu Chemical Co., Ltd. at Tokyo, Japan. If KSG-18 is used in the composition of the present invention as the gellant, it may present in an amount ranging from 35% to about 55%, more preferably about 40% to about 50%, and most preferably from about 44% to about 46%, by total weight of each composition.

The transparent cosmetic composition of the present invention as described hereinabove is preferably anhydrous, i.e., containing less than about 0.5% of water by total weight of the composition, and alcohol-free, i.e., containing less than about 0.5% of ethyl alcohol by total weight of the composition. More preferably, the composition of the present invention is in form of a gel emulsion and has a viscosity in the range of 16,000 cps to 28,000 cps.

Other biological active agents can be added to the present inventive compositions as desired, as long as the clarity and viscosity are not compromised. Routine experimentation can determine the amounts required to retain a stable composition. The type of biological active agent added, can be any which is beneficially used in a topical cosmetic or pharmaceutical composition. For example, the composition can further comprise moisturizing actives, agents used to treat age spots, keratoses and wrinkles, as well as analgesics, anesthetics, anti-acne agents, antibacterials, antiyeast agents, antifungal agents, antiviral agents, antidandruff agents, antidermatitis agents, antipruritic agents, antiemetics, antimotion sickness agents, anti-irritant agents, anti-inflammatory agents, antihyperkeratolytic agents, anti-dry skin agents, antipsoriatic agents, antiseborrheic agents, antiaging agents, antiwrinkle agents, additional sunscreen agents, antihistamine agents, skin lightening agents, depigmenting agents, wound-healing agents, vitamins, corticosteroids, self-tanning agents, or hormones. The above-described biological active agents are only optional components of the sunscreen composition of the present invention and may be omitted from such composition without materially affecting the sun-screening function of the composition.

The sunscreen composition of the present application further comprises a cosmetically acceptable vehicle, as long as the clarity and viscosity are not compromised. Substances which may be formulated into the sunscreen composition of the present application include, but are not limited to: moisturizing agents, astringent agents, chelating agents, surfactants, emollients, preservatives, stabilizers, humectants, fragrances, etc. Preferably, but not necessarily, such vehicles aid the formation of a protective layer on the skin. The vehicle or vehicles can present in the topical composition of the present invention at an amount ranging from about 1% to about 99.9%, preferably from about 50% to about 99.5%, more preferably from about 70% to about 99%, and most preferably from about 80% to 90% by total weight of the topical composition.

For example, emollients which may be used in the sunscreen composition of the present invention include, but are not limited to: stearyl alcohol, cetyl alcohol, oleyl alcohol, isocetyl alcohol, fatty alcohols, propane-1,2-diol, butane-1,3-diol, octadecan-2-ol, glyceryl monostearate, isopropyl isostearate, stearic acid, isostearic acid, isocetyl stearate, isopropyl stearate, butyl stearate, isopropyl laurate, hexyl laurate, decyl oleate, isobutyl palmitate, cetyl palmitate, isopropyl palmitate, palmitic acid, dimethylpolysiloxane, glyceryl monoricinoleate, di-n-butyl sebacate, isopropyl myristate, butyl myristate, myristyl myristate, isopropyl linoleate, lauryl lactate, myristyl lactate, polyethylene glycol, triethylene glycol, lanoline, acetylated lanolin, sesame oil, coconut oil, arrachis oil, castor oil, mink oil, mineral oil, and petroleum.

A variety of preservatives can also be added to the sunscreen compositions of the present invention to provide a prolonged shelf life. Suitable preservatives include, but are not limited to: potassium sorbate, imidazolidinyl urea, p-hydroxy benzoate, esters of p-hydroxybenzoic acid, CTFA designation parabens, ethylhexylglycerin, caprylyl glycol/phenoxyethanol/hexylene glycol, etc. Other preservatives suitable for use in the sunscreen compositions of the present invention are disclosed in the International Cosmetic Ingredient Dictionary and Handbook, twelfth edition, 2004, the entire disclosure of which is herein incorporated by reference.

Humectants which may be used include, but are not limited to: polyhydric alcohols including glycerol, polyalkylene glycols, and alkylene polyols and mixtures thereof, hyaluronic acid, urea, glycerin, sorbitol, sodium 2-pyrrolidone-5-carboxylate, soluble collagen, dibutylphthalate and gelatin.

The transparent sunscreen composition of the present invention may optionally comprise a fragrance in an amount sufficient to make the composition more appealing to the consumer. Preferably, the fragrance is in the amount of from about 0.01% to about 10% by total weight of the composition.

The above-described sunscreen compositions are particularly useful as skin-protecting or skin-treatment products for protecting the human skin against the harmful effect of UV radiation. Such sunscreen compositions can be applied to eyes, cheek, chin, neck and other facial areas, and it can also be applied to other bodily areas that are potentially susceptible to UV radiation.

The methods of use for the sunscreen compositions of the present invention will depend on the ultimate intended use of composition. For example, the sunscreen composition of the present invention may be applied to the skin on an as-needed basis to provide occasional protection against particularly intense sun light exposures. Alternatively and preferably, the sunscreen composition is applied to the skin in a regularly manner. For example, the sunscreen composition can be applied regularly at a frequency ranging from about once per week to about three times per day.

The following examples further illustrate the novel compositions and methods of the present invention, but the invention is not limited thereto.

### EXAMPLE 1

The following compositions provide examples of several preferred embodiments of the present invention in the form of emulsion gels.

| | | |
|---|---|---|
| | | CONC. |
| TRADE NAME | CTFA NAME | (wt. %) |

Composition A

| | | |
|---|---|---|
| Parsol MCX/ESCALOL 557 | Ethylhexyl Methoxycinnamate | 7.5000 |
| Phospholipon 90-H | Hydrogenated Lecithin | 0.1000 |
| Poly-Pore 350FA | Allyl Methacrylates Crosspolymer Ferulic Acid | 0.3000 |
| Nordihydroguaiaretic Acid | Nordihydroguaiaretic acid | 0.0500 |
| Rosmarinic Acid 90% | Rosmarinus Officinalis (Rosemary) Extract | 0.0500 |
| Neo Heliopan OS | Ethylhexyl Salicylate | 5.0000 |
| Eusolex HMS | Homosalate | 5.0000 |
| Lexfeel 7 | Neopentyl Glycol Diheptanoate | 3.0000 |
| Parsol 1789 | Avobenzone | 2.0000 |
| DM-Fluid A-6CS | Dimethicone | 10.999 |
| Liponyl Yeast AE | Nylon 6/12//Yeast Extract (FAEX) | 0.1000 |
| Amentoflavone | Selaginella Tamariscina (Spike Moss) Extract | 0.0010 |
| 1,3 Butylene Glycol | Butylene Glycol | 0.5000 |
| X-21-5652D | Methyl Trimethicone//Dimethicone/Phenyl Vinyl Dimethicone | 61.000 |
| | crosspolymer//Diphenylsioloxy Phenyl Trimethicone | |
| KSP-100 | Vinyl Dimethicone/Methicone Silsesquioxane Crosspolymer | 3.5000 |
| Capmul 907P | Propylene Glycol Heptanoate | 0.6000 |
| Castor Oil Ext. (30% Yellow 5 | Castor Oil Ext. (30% Yellow 5 B3397) | 0.2300 |
| B3397) | | |
| Castor Oil Ext. (30% RED 28 | Castor Oil Ext. (30% RED 28 C6529) | 0.2300 |
| C6529) | | |

Composition B

| | | |
|---|---|---|
| Parsol MCX/ESCALOL 557 | Ethylhexyl Methoxycinnamate | 7.5000 |
| Phospholipon 90-H | Hydrogenated Lecithin | 0.1000 |
| Poly-Pore 350FA | Allyl Methacrylates Crosspolymer Ferulic Acid | 0.3000 |
| Nordihydroguaiaretic Acid | Nordihydroguaiaretic acid | 0.0500 |
| Rosmarinic Acid 90% | Rosmarinus Officinalis (Rosemary) Extract | 0.0500 |
| Neo Heliopan OS | Ethylhexyl Salicylate | 7.0000 |
| Eusolex HMS | Homosalate | 3.0000 |
| Lexfeel 7 | Neopentyl Glycol Diheptanoate | 3.0000 |
| Parsol 1789 | Avobenzone | 2.0000 |
| DM-Fluid A-6CS | Dimethicone | 10.999 |
| Liponyl Yeast AE | Nylon 6/12//Yeast Extract (FAEX) | 0.1000 |
| Amentoflavone | Selaginella Tamariscina (Spike Moss) Extract | 0.0010 |
| 1,3 Butylene Glycol | Butylene Glycol | 0.5000 |
| KSG-18A | Diphenylsiloxy Phenyl Trimethicone//Dimethicone/Phenyl Vinyl | 46.000 |
| | Dimethicone Crosspolymer | |
| X-21-5652D | Methyl Trimethicone//Dimethicone/Phenyl Vinyl Dimethicone | 15.000 |
| | crosspolymer//Diphenylsioloxy Phenyl Trimethicone | |
| KSP-100 | Vinyl Dimethicone/Methicone Silsesquioxane Crosspolymer | 3.5000 |
| Phenoxetol | Phenoxyethanol | 0.3000 |
| Capmul 907P | Propylene Glycol Heptanoate | 0.3000 |
| Castor Oil Ext. (30% Yellow 5 | Castor Oil Ext. (30% Yellow 5 B3397) | 0.2300 |
| B3397) | | |
| Castor Oil Ext. (30% RED 28 | Castor Oil Ext. (30% RED 28 C6529) | 0.0700 |
| C6529) | | |

Composition C

| | | |
|---|---|---|
| Parsol MCX/ESCALOL 557 | Ethylhexyl Methoxycinnamate | 7.5000 |
| Neo Heliopan OS | Ethylhexyl Salicylate | 7.0000 |
| Eusolex HMS | Homosalate | 3.0000 |
| Lexfeel 7 | Neopentyl Glycol Diheptanoate | 3.0000 |
| Parsol 1789 | Avobenzone | 2.0000 |
| DM-Fluid A-6CS | Dimethicone | 12.628 |
| KSG-18 | Phenyl Trimethicone//Dimethicone/Phenyl Vinyl Dimethicone | 46.500 |
| | Crosspolymer | |
| X-21-5652D | Methyl Trimethicone//Dimethicone/Phenyl Vinyl Dimethicone | 15.000 |
| | crosspolymer//Diphenlsioloxy Phenyl Trimethicone | |
| KSP-100 | Vinyl Dimethicone/Methicone Silsesquioxane Crosspolymer | 2.5000 |
| Capmul 907P | Propylene Glycol Heptanoate | 0.3000 |
| Castor Oil Ext. (30% Yellow 5 | Castor Oil Ext. (30% Yellow 5 B3397) | 0.2300 |
| B3397) | | |
| Castor Oil Ext. (30% RED 28 | Castor Oil Ext. (30% RED 28 C6529) | 0.0420 |
| C6529) | | |
| Phenoxetol | Phenoxyethanol | 0.3000 |

### EXAMPLE 2

The following example shows comparative results of compositions using varying gelling materials (marked in bold). All of the ingredients were mixed together and observed over a 24 hour period for clarity and stability.

TABLE 1

| TRADE NAME | CTFA NAME | 1 | 2 | 3 | 4 | 5 |
|---|---|---|---|---|---|---|
| Parsol MCX | Ethylhexyl Methoxycinnamate | 7.5 | 7.5 | 7.5 | 7.5 | 7.5 |
| Neo Heliopen | Ethylhexyl Salicylate | 5 | 5 | 5 | 5 | 5 |
| Eusolex | Homosalate | 5 | 5 | 5 | 5 | 5 |
| Parsol 1789 | Avobenzone | 2 | 2 | 2 | 2 | 2 |
| KSG-18 | Phenyl | 65.5 | 80.5 | 0 | 0 | 0 |
| | Trimethicone//Dimethicone/Phenyl | | | | | |
| | Vinyl Dimethicone Crosspolymer | | | | | |
| | gel | | | | | |
| X-21-5652D | Methyl | 15 | 0 | 0 | 0 | 80.5 |
| | Trimethicone//Dimethicone/Phenyl | | | | | |
| | Vinyl Dimethicone | | | | | |
| | crosspolymer//Diphenylsioloxy | | | | | |
| | Phenyl Trimethicone | | | | | |
| Gransil DM5 | Dimethicone gel | 0 | 0 | 80.5 | 0 | 0 |
| Gransil TMG | Cyclopentasiloxane gel | 0 | 0 | 0 | 80.5 | 0 |

| | | | | | |
|---|---|---|---|---|---|
| Observations | clear | not clear, | not clear | not clear | clear |
| | | unstable | | | |

As seen from Table 1 above, the compositions comprising the inventive diphenyl silicone elastomer gellant (i.e., the X-21-5652D gellant) were the only compositions that remained clear with sufficient stability.

### EXAMPLE 3

The following example shows comparative results of compositions containing various different solvents in the base composition 1 of Example 2 described hereinabove. Each solvent was mixed into the base composition 1 of Example 2 and observed over a 24 hour period for clarity and stability

TABLE 2

| SAMPLE# | SOLVENT NAME | SOLVENT % | OBSERVATIONS |
|---|---|---|---|
| 1 | Dimethicone | 5 | Ok |
| 2 | Cetiol CC | 5 | Ok |
| 3 | Ceraaphyl 31 | 5 | Ok |
| 4 | Lexfeel 7 | 5 | Ok |
| 5 | Glycerine | 5 | Ok |
| 6 | Cetiol HE | 12 | 50C unstable |
| 7 | Olive oil | 12 | Stable but not a gel |
| 8 | Hydrovance | 12 | Not stable |
| 9 | Fitoderm | 12 | Stable but not a gel |
| 10 | Ceraphyl | 12 | Stable but not a gel |

As seen from Table 2 above, only certain specific solvents provide stable, clear gels, while others solvents either destroy stability of the composition or do not create a gel composition.

What is claimed is:

1. A cosmetic composition comprising:
at least one chemical ultra-violet (UV) light absorber;
at least one solvent selected from the group consisting of dimethicone, dicaprylyl carbonate, lauryl lactate, neopentyl glycol diheptanoate, glycerin, and combinations thereof; and
at least one diphenyl silicone elastomer gellant comprising diphenylsiloxy phenyl trimethicone,
wherein the composition is transparent and is essentially free of alcohol.

2. The composition of claim 1, wherein the chemical UV light absorber is selected from the group consisting of benzophenone-3, homosalate, ethylhexyl methoxycinnamate, avobenzone, ethylhexyl salicylate, octocrylene and combinations thereof.

3. The composition of claim 1, wherein the diphenylsiloxy phenyl trimethicone is present at a concentration ranging from about 1% to about 50% by total weight of the composition.

4. The composition of claim 1, wherein the diphenyl silicone elastomer gellant comprises a mixture of diphenylsiloxy phenyl trimethicone and dimethicone/phenyl vinyl dimethicone crosspolymer.

5. The composition of claim 1, wherein the diphenyl silicone elastomer gellant comprises a mixture of diphenylsiloxy phenyl trimethicone, dimethicone/phenyl vinyl dimethicone crosspolymer, and methyl trimethicone.

6. The composition of claim 1, wherein the diphenyl silicone elastomer gellant comprises a first mixture of diphenylsiloxy phenyl trimethicone and dimethicone/phenyl vinyl dimethicone crosspolymer, and a second mixture of diphenylsiloxy phenyl trimethicone, dimethicone/phenyl vinyl dimethicone crosspolymer, and methyl trimethicone.

7. The composition of claim 1, further comprising a hybrid silicone powder with a silicone rubber core and a silicone resin shell.

8. The composition of claim 7, wherein the hybrid silicone powder comprises a vinyl dimethicone/methicone silsesquioxane crosspolymer.

9. The composition of claim 1, further comprising a thickener comprising a dimethicone/phenyl vinyl dimethicone crosspolymer.

10. The composition of claim 9, wherein the thickener comprises a mixture of the dimethicone/phenyl vinyl dimethicone crosspolymer with phenyl trimethicone.

11. A method of protecting skin against harmful effects of ultra-violet (UV) radiation, comprising the step of applying to the skin a transparent cosmetic composition comprising at least one chemical UV light absorber, at least one solvent selected from the group consisting of dimethicone, dicaprylyl carbonate, lauryl lactate, neopentyl glycol diheptanoate, glycerin, and combinations thereof, and at least one diphenyl silicone elastomer gallant comprising diphenylsiloxy phenyl trimethicone, wherein said transparent cosmetic composition is essentially free of alcohol.

12. The method of claim 11, wherein the chemical UV light absorber is selected from the group consisting of benzophenone-3, homosalate, ethylhexyl methoxycinnamate, avobenzone, ethylhexyl salicylate, octocrylene and combinations thereof.

13. The method of claim 11, wherein the diphenylsiloxy phenyl trimethicone is present at a concentration ranging from about 1% to about 50% by total weight of the composition.

14. The method of claim 11, wherein the diphenyl silicone elastomer gellant comprises a mixture of diphenylsiloxy phenyl trimethicone and dimethicone/phenyl vinyl dimethicone crosspolymer.

15. The method of claim 11, wherein the diphenyl silicone elastomer gellant comprises a mixture of diphenylsiloxy phenyl trimethicone, dimethicone/phenyl vinyl dimethicone crosspolymer, and methyl trimethicone.

16. The method of claim 11, wherein said diphenyl silicone elastomer gellant comprises a first mixture of diphenylsiloxy phenyl trimethicone and dimethicone/phenyl vinyl dimethicone crosspolymer, and a second mixture of diphenylsiloxy phenyl trimethicone, dimethicone/phenyl vinyl dimethicone crosspolymer, and methyl trimethicone.

17. The method of claim 11, wherein said transparent cosmetic composition further comprises a hybrid silicone powder with a silicone rubber core and a silicone resin shell.

18. The method of claim 17, wherein the hybrid silicone powder comprises a vinyl dimethicone/methicone silsesquioxane crosspolymer.

19. The method of claim 11, wherein said transparent cosmetic composition further comprises a thickener comprising a dimethicone/phenyl vinyl dimethicone crosspolymer.

20. The method of claim 19, wherein the thickener comprises a mixture of the dimethicone/phenyl vinyl dimethicone crosspolymer with phenyl trimethicone.
